(19) United States
(12) Patent Application Publication
(10) Pub. No.: US 2006/0127344 A1
(43) Pub. Date: Jun. 15, 2006

(54) Novel cosmetic emulsions and emulsifiers exhibiting dilatant rheological properties

(76) Inventors: Albert Zofchak, Holmdel, NJ (US); Paul Slavashevich, Bronx, NY (US); John Carson, Union City, NJ (US)

Correspondence Address: COLEMAN SUDOL SAPONE, P.C., 714 COLORADO AVENUE, BRIDGE PORT, CT, 06605-1601, US

(21) Appl. No.: 11/302,667

(22) Filed: Dec. 14, 2005

Related U.S. Application Data
(60) Provisional application No. 60/636,307, filed on Dec. 15, 2004.

(51) Int. Cl. A61K 8/42

(52) U.S. Cl. 424/70.31

(57) ABSTRACT

The use of novel nonionic polymeric thickeners that produce cosmetically useful emulsions of an oil phase and a water phase is described. The thickeners of choice are the urethane dimers of ethoxylated fatty alcohols. These materials were not anticipated to have emulsifying properties. Also, it has been found that the esters of ethoxylated polyols show similar emulsifying behavior, although their uses are limited by their inherent susceptibility towards hydrolysis. Further, many of the emulsions produced with these emulsifiers have a dilatant or shear thickening rheology that is unusual and has unique cosmetic applications. In addition, it is possible to produce high internal phase oil-in-water emulsions, with oil phase concentrations greater than 70%, using these emulsifiers. Finally, many of the emulsions, especially the high internal phase ones, have a characteristic “ring” or vibration that is apparent when their container is tapped. This is a characteristic of so called “microemulsions” that has not been observed in conventional appearing, white (albeit dilatant) emulsions.

### FIELD OF THE INVENTION

This invention relates to cosmetics and toiletries. More specifically, it refers to the preparation of hair and skin care products that are emulsions of oil in water. Emulsions are widely used in personal care products to moisturize, protect and improve the condition and feel of skin and hair. In addition, emulsions provide aesthetically pleasing vehicles for the delivery of topical medicaments and can be used to provide the simultaneous delivery of oil and water soluble materials. Further, in hair care products, emulsions are used to improve the styling and appearance properties of hair such as shine, combability, style hold and hair feel. The present emulsions provide ringing and novel dilatant properties, which may be used effectively in lotions, salves and other personal care products to be placed onto the skin and/or hair.

### BACKGROUND AND RELATED ASPECTS OF THE INVENTION

Emulsions have a long and established history of use as moisturizing and healing preparations in skin creams and lotions. They are also used to deliver ingredients to hair to improve its condition. Typically, the emulsion used in cosmetic products is an oil in water (o/w) type emulsion in which an oil (water insoluble liquid material) is dispersed as discrete droplets in an external, continuous water phase. The dispersion is usually accomplished by using a surfactant which lowers the interfacial forces between the water and the oil (thus facilitating the dispersion of the oil within the water) and provides a means to stabilize the dispersion (usually by creating a barrier substance that discourages the close approach and coalescence of the dispersed phase droplets). Surfactants are molecules that have dual functionality in that they have both water compatible and oil compatible portions. This allows them to be soluble (and compatible to some degree) in both phases of an emulsion. The water soluble portion of the surfactant molecule can be charged, or it can be nonionic. One of the most common methods used to make a nonionic surfactant is via the ethoxylation of a fatty acid, a fatty alcohol or a fatty 1° or 2° amine. The greater the number of moles of ethylene oxide that are reacted onto the base compound (i.e. a fatty alcohol), the more water soluble the resulting surfactant becomes. Often the number of moles of ethylene oxide is 20 or more, thus, these materials can be thought of as oxyethylene polymers. Interestingly, it has been found that if the hydroxy group on the terminal hydroxyethylene group is reacted (i.e. esterified), then the material loses water solubility and becomes a poorer emulsifier. For example: a 75 mole ethoxylate of stearyl alcohol (Steareth-75) is a very water soluble surfactant that is used to solubilize oils and will make clear dispersions of oils in water. However, the distearate of a 150 mole polymer of ethylene oxide (PEG-150 distearate) makes a hazy/cloudy solution in water, has some o/w emulsifying ability and will make a typically appearing white, opaque emulsion with mineral oil but it does not make clear dispersions of oils in water. This material usually finds use as a viscosity builder in surfactant systems that can clarify and solubilize it.

Within the recent past, new polymeric o/w emulsifiers, based upon various structures, have become available. These materials generally consist of a water soluble polymer backbone with pendant fatty groups. The fatty groups function similarly to the oil soluble portion of a conventional surfactant, in that, they interact with the oily material in an emulsion to help solubilize or compatibilize it with water, while the water soluble portion remains in solution in the water. This has the same effect as that of a conventional surfactant, i.e. making the oil and water phases compatible. In addition, the water soluble portion (because it is a large molecular weight polymer) forms a hydrated layer in the water phase that surrounds the oil droplets. This layer acts as a barrier to the close approach and coalescence of the dispersed oil phase droplets, thus stabilizing the emulsion.

The advantages of polymeric surfactants/emulsifiers are at least threefold: 1) in general, they are extremely mild and non-irritating to skin and mucous membranes—because they are large molecules, they can not penetrate into the skin to cause irritation; 2) concentrations of these surfactants are generally quite low which further reduces the irritation potential of emulsions made with these polymers; and 3) once a polymeric surfactant based emulsion is applied to a surface and dried, the polymeric emulsifier, generally, becomes poorly functional and has little ability to re-emulsify the oil phase from the surface. This is in contrast to conventional o/w emulsifiers that remain readily water soluble and are quite able to re-emulsify the applied oils. The above-described properties make polymeric surfactants extremely useful for formulating waterproof sunscreen emulsions that resist wash off while the wearer is swimming. The disadvantages of polymeric surfactants are minimal, but can include: occasional difficulty in dispersing and hydrating the polymer, and the occasional need to add an auxiliary or secondary water soluble wetting agent/emulsifier to help initiate emulsification and to establish the oil droplet particle size.

Several types of polymeric emulsifiers are available commercially. The anionic types tend to be more widely used in commercial personal care products. These are typified by the Pemulen® products and have the INCI name of Acrylates C10-30 Alkyl Acrylates Crosspolymer. The use level is from about 0.1% to about 1.0%. The products are sold as 100% active powders. The powder is usually dispersed in the water phase prior to the addition of the oil phase. Alternatively, the polymer powder can be dispersed in the oil phase prior to its addition to the water phase. Once the two phases are combined, the polymer is neutralized with a base. This causes dramatic thickening and completes the emulsion. Another commonly used type of polymeric surfactant is Alkyl modified Hydroxyethyl Cellulose such as Natrosol® Plus CS (INCI name—Cetyl Hydroxyethyl Cellulose). These polymers are typically dispersed and hydrated in the water phase, prior to the addition of the oil phase. The third type of polymeric emulsifier has a cationic charge and is typified by the Polylipid B emulsifier (INCI Name—TEA Behenate Ethomonium IPDI Ethosulfate). These cationic emulsifiers also do not need neutralization and are generally added to the oil phase of an emulsion prior to its addition to the water phase.

The emulsions made with these “conventional” polymeric emulsifiers generally have a shear thinning rheology. That is, they become less viscous and flow more readily when rubbed on the skin. Dilatant (or shear thickening) emulsions, on the other hand, become thicker when rubbed on skin and thus, resist spreading. This characteristic is generally not well appreciated by consumers or marketers and few, if any, dilatant personal care products are available, because they can be difficult to apply. Their characteristics are distinct and quite opposite to those of the conventional chemistries. But, there are applications and products where this type of emulsion would have appeal because the characteristics of these emulsions are new and unique in form, feel and function. In addition, the emulsions of the present invention, even though they are dilatant, readily release their internal oil phase when rubbed on the skin. This may be caused by the emulsion structure “breaking down” when rubbed or by the loss of water from the emulsion as it is rubbed on the skin. But whatever the cause, this “oil phase release” facilitates the application of the emulsions of the present invention upon the skin. Thus, the emulsion does not simply “ball up” and roll around without any contact or spreading when it is rubbed on the skin.

Most emulsions (with the exception of microemulsions) do not “ring” when tapped. This “ring” is a noticeable vibration that occurs in the emulsion when the emulsion's container is tapped. Ringing is a characteristic that is usually associated with solid, microemulsion gels and it is not seen in typical opaque, shear thinning emulsions. In the instances noted here, the cause of the ringing may be the dilatancy or it may be due a structure created in the water phase by the emulsifying polymers of this invention. But, whatever the cause, it is an unanticipated effect that adds interest and a “point of difference” to these types of emulsions. Finally, most emulsions have limits upon the amount of oil (or internal phase) that they can incorporate. Emulsions of the present invention can contain from as little as a few percent oil phase to about 90%. In contrast to conventional emulsifiers, the use concentration of the emulsifiers of the present invention, decreases as the concentration of the internal phase increases. Thus, while 3.5% of an emulsifier of the present invention may be needed to emulsify 50% mineral oil in water, only about 1% or even less of the emulsifier may needed to make a 90% mineral oil emulsion. This may be because the mechanism of emulsification of the emulsifiers of the present invention is one of “gelling” the water phase, thus preventing the close approach and coalescence of the dispersed phase as well as being a surfactant that reduces interfacial tension.

Thus, the current invention deals with the formation of o/w emulsions that have a unique dilatant rheology and are created through the use of nonionic polymeric surfactants in novel emulsions according to the present invention. Specific properties, formulations, methods for modifying the emulsion properties and suggested applications for these unique emulsions are described.

### OBJECTS OF THE INVENTION

It is an object of the invention to provide novel polymeric surfactants/emulsifiers which may be optimally used to form emulsions to be used in personal care product compositions.

It is an additional object of the invention to provide emulsions and other compositions which may be used to produce personal care products/compositions according to the present invention which have unexpected dilatancy properties.

It is yet another object of the invention to provide personal care compositions which contain effective amounts or concentrations of the present surfactant/emulsifier compositions to provide emulsions and/or other compositions as components for personal care compositions or as final products.

It is still a further object of the invention to provide methods for producing polymeric surfactants/emulsifiers, emulsions and/or final personal care compositions according to the present invention.

It is an additional object of the present invention to provide shear thickening (dilatancy) emulsions.

It is a further object of the present invention to provide high internal phase (greater than about 75%) o/w emulsions using polymeric emulsifiers.

It is still an additional object of the present invention to control the rheology of the present emulsions by controlling any one or more of the following characteristics: 1) controlling the amount and nature of the internal phase (oily) material that is emulsified; 2) controlling the amount and the types of the nonionic polymeric emulsifiers of the present invention that are used; 3) controlling the amount and types of additional (secondary) surfactants, polymeric surfactants and water or oil soluble polymers that are included in the present compositions; 4) controlling the amount and type of solvents, diols or polyols that are used to modify the emulsion's rheology; and/or 5) controlling the amount and type of solid materials such as, but not limited to, talc, silica, alumina, pigments, solid organic polymers and waxes that are used to modify the emulsion's rheology.

Any one or more of these and/or other objects of the invention may be readily gleaned from a review of the specification which follows.

### SUMMARY OF THE INVENTION

The present invention relates to novel emulsion compositions which exhibit unexpected dilatancy properties. The emulsions according to the present invention comprise an oil, water and an emulsifier or surfactant as otherwise described hereinbelow, and optionally, a number of other components which may be added to the compositions in order to instill various characteristics of traditional personal care products, including, for example, solvents, coloring agents, secondary emulsifiers, humectants, moisturizing agents, pigments, anti-perspirant agents, aromatic or deodorizing agents, uv-absorbing compounds (for example, as used in sunscreens), preservatives, skin and/or hair conditioning agents, hair-straightening agents and the like, as otherwise discussed in detail herein. Compositions according to the present invention may be adapted for use on the skin and hair, and are particularly useful for delivering components to the skin which take advantage of the present compositions' dilatancy properties.

In general terms, compositions according to the present invention related broadly to emulsions comprising about 5% to about 90% by weight of an oil, about 10% to about 95% by weight water and about 0.1% to about 20% by weight of an emulsifier (preferably about 1% to about 10% of an emulsifier, even more preferably about 1% to about 5% of an emsulsifier within this range which varies as a function of the amount of oil and water within the composition), and optionally, about 0% to about 10% (preferably, at least about 0.1% to about 5% within this range) by weight of a secondary emulsifier, about 0% to about 20% by weight of a solvent (preferably, about 0.1% to about 10% by weight, preferably, about 2% to about 5% by weight, depending upon solvent and the amount of oil, water, emulsifier and type of emulsifier used in the composition noting that higher weight % of solvent may negatively impact the dilatancy characteristics of the composition, making the composition thinner and less viscous as the weight percent of solvent increases.

The polymeric surfactants which are used in the present invention are non-ionic polyoxyethylene compounds having fatty (hydrophobic) residues at distill ends of each polyoxyetheylene chain. The surfactants according to the present invention include several types and chemistries, but all employ polyoxyethylene as the water soluble portion of the molecule. The most elementary example of the type of material that is described in this invention as a polymeric surfactant emulsifier is the preferred surfactantlemulsifier PEG-150 distearate. This material is commonly used in personal care products, especially cleaning products such as shampoos and shower gels as a viscosity builder. The emulsions that it does make can have a rheology that varies from shear thinning to dilatant. Dilatant emulsions according to the present invention are those which are preferred for use in the present invention. While there is interest in these emulsions, the major interest is in the dilatant emulsions.

Surfactants according to the present invention which may be used to create emulsions according to the present invention comprise compounds according to the following structures I, II and III: 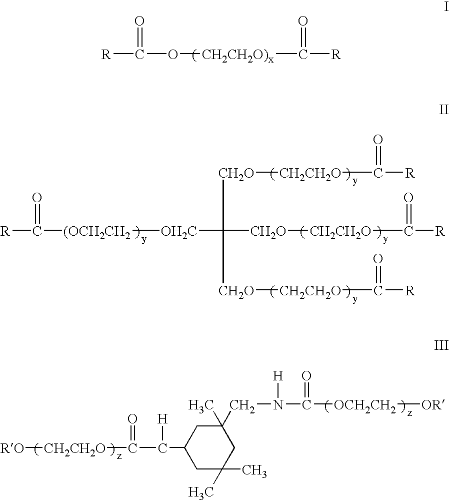
wherein x is about 150; y is about 35 to about 40 such that the total of 4y is about 150 and z is about 75-100, preferably 75 or 100; and
Each R is independently a C₁₅-C₂₁ alkyl or alkene group, preferably an alkyl group, more preferably a C₁₇ alkyl group; and each R′ is independently a C ₁₆-C₂₂ alkyl or alkene group, preferably an alkyl group, more preferably a C₁₈ alkyl group.

Thus, it is an objective, but not a necessary condition, to produce shear thickening emulsions (emulsions with dilatancy properties). It is an objective, but not a necessary condition of this invention to be able to make high internal phase (greater than 75%) o/w emulsions using polymeric emulsifiers. Emulsifiers that are capable of making such high internal phase emulsion include: PEG-150 Distearate, Crothix (INCI Name: PEG-150 Pentaerythrityl Tetrastearate), Dermothix 75 (INCI Name: Disteareth-75 IPDI) and Dermothix 100 (INCI Name: Disteareth-100 IPDI) and their combinations.

It is an objective of this invention to make emulsions for personal care use, that employ the nonionic polymeric emulsifiers mentioned above or described herein, that are shear thickening (exhibit dilatancy) as opposed to shear thinning. Such emulsions can have significant tactile, visual and product application appeal because of their unique rheology. The emulsions find use as skin creams and lotions, sunscreens, antiperspirants, make up products, mascaras, hair conditioners, hair styling aids, hair bleaches, hair relaxers, hair dyes and vehicles for medicaments.

It is an objective of this invention to be able to control the rheology of these shear thickening emulsions by 1) controlling the amount and nature of the internal phase (oily) material that is emulsified, 2) controlling the amount and the types of the nonionic polymeric emulsifiers of the present invention that are used, 3) controlling the amount and types of additional surfactants, polymeric surfactants and water or oil soluble polymers that are used 4) controlling the amount and type of solvents, diols or polyols that are used to modify the emulsion's rheology and 5) controlling the amount and type of solid materials such as, but not limited to, talc, silica, alumina, pigments, solid organic polymers and waxes that are used to modify the emulsion's rheology.

### DETAILED DESCRIPTION OF THE INVENTION

The following terms shall be used throughout the specification to describe the present invention.

The terms “personal care product” and “personal care composition” are used synonymously to describe chemical compositions according to the present invention which may be used on the hair, skin and/or nails of the user and include hair products such as shampoos, hair coloring and conditioning agents, cosmetic compositions, nail care and various sunscreens and deodorants/antiperspirants and various skin car products, among others. Note that emulsions according to the present invention may also be used as topical delivery vehicles for bioactive agents.

The term “surfactant” or “emulsifier” are used synonymously throughout the specification to describe compounds according to the present invention which contain both hydrophilic and hydrophobic residues and can be used to produce emulsions with an “oil” and water. These compounds/compositions are described in detail in the present application.

The term “effective” is used generally to describe amounts of compounds according to the present invention which are added to compositions to produce an intended effect.

Surfactant/emulsifier compounds according to the present invention exhibit advantageous features which are related to their chemical structure. For example, surface active agents according to the present invention are advantageous for their dilatant properties, thus making certain aspects of emulsions and personal care products produced with these compounds unique in their textural feel and delivery of active components within an oil phase of the emulsion. These agents are particularly useful for formulating personal care products, especially sunscreens, handcreams, soaps, shaving creams/gels, deodorants/antiperspirants, and other products which make use of the compounds' ability to exhibit increased viscosity and the compositions are rubbed into the skin. Noted here is the fact that surfactant compositions according to the present invention are polymeric in nature. As noted, a polymeric surfactant according to the present invention (at least as to that portion of the molecule which is polymeric in nature) is described according to the number of monomeric units which are polymerized to produce the polymeric structure, which is based upon an average molecular weight of a resulting polymer which is produced. One of ordinary skill in the art will recognize that the number of moles of a monomer which is used to describe a polymeric structure is not absolute in value, but represents a mean number of moles polymerized across a typical Gaussian distribution of molecules.

The terms “emulsion” and “oil-in-water emulsion” are used throughout the specification to describe certain compositions according to the present invention which contain in its broadest aspect, a water phase, an oil phase and an emulsfier. An “emulsion” according to the present invention is a cream or lotion which is generally formed by the suspension of a very finely divided liquid, in this case an oil, in another liquid, in this case, water. In the present invention, an oil-in-water emulsion is formed when the oil phase is compatabilized in the water phase such as that the oil phase becomes hidden within the water phase. While not being limited by way of theory, it is believed that in the emulsion compositions according to the present invention, the water phase produces an encapsulation-like structure or a related structure surrounding an oil phase (oil-in-water emulsion), with the novel surfactants/emulsifiers of the present invention serving to cause or enhance the formation of the emulsion composition. The term emulsion is used to distinguish the present compositions from compositions which contain at least two distinct phases, i.e., an oil phase and a water phase. The present surfactants/emulsifiers according to the present invention exhibit primary emulsifier activity in oil-in-water emulsions when used alone or in combination with secondary or auxiliary emulsifiers.

The term “oil” is used throughout the specification to describe any of various lubricious, hydrophobic and combustible substances obtained from animal, vegetable and mineral matter, which may be used to form oil-in-water emulsions according to the present invention. Oils for use in the present invention may include petroleum-based oil derivatives such as purified petrolatum and mineral oil. Petroleum-derived oils include aliphatic or wax-based oils, aromatic or asphalt-based oils and mixed base oils and may include relatively polar and non-polar oils. “Non-polar” oils are generally oils such as petrolatum or mineral oil or its derivatives which are hydrocarbons and are more hydrophobic and lipophilic compared to synthetic oils, such as esters, which may be referred to as “polar” oils. It is understood that within the class of oils, that the use of the terms “non-polar” and “polar” are relative within this very hydrophobic and lipophilic class, and all of the oils tend to be much more hydrophobic and lipophilic than the water phase which is used in the present invention. Exemplary non-polar synthetic oils include, for example, silicones, dimethicones and cyclomethicones, among others, and fluorocarbons such as CF-76 (a volatile fluorocarbon).

In addition to the above-described oils, certain essential oils derived from plants such as volatile liquids derived from flowers, stems and leaves and other parts of the plant which may include terpenoids and other natural products including triglycerides may also be considered oils for purposes of the present invention.

Petrolatum (mineral fat, petroleum jelly or mineral jelly) and mineral oil products for use in the present invention may be obtained from a variety of suppliers. These products may range widely in viscosity and other physical and chemical characteristics such as molecular weight and purity. Preferred petrolatum and mineral oil for use in the present invention are those which exhibit significant utility in cosmetic and pharmaceutical products. Cosmetic grade oils are preferred oils for use in the present invention.

Additional oils for use in the present invention may include, for example, mono-, di- and tri-glycerides (glyceride esters) which may be natural or synthetic (derived from esterification of glycerol and at least one organic acid, saturated or unsaturated, such as for example, such as butyric, caproic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds or nuts and include drying oils, for example, linseed, iticica and tung, among others; semi-drying oils, for example, soybean, sunflower, safflower and cottonseed oil; non-drying oils, for example castor and coconut oil; and other oils, such as those used in soap, for example palm oil. Hydrogenated vegetable oils also may be used in the present invention. Animal oils are also contemplated for use as glyceride esters and include, for example, fats such as tallow, lard and stearin and liquid fats, such as fish oils, fish-liver oils and other animal oils, including sperm oil, among numerous others. In addition, a number of other oils may be used, including C₁₂-C₃₀ (or higher) fatty esters (other than the glyceride esters, which are described above) or any other acceptable cosmetic emollient. These may be natural or synthetic.

Preferred oils for use in the present invention include petrolatum, mineral oil or mixtures of petrolatum and mineral oil where the amount of petrolatum to mineral oil (on a weight/weight basis) ranges from about 1:20 to about 10:1, preferably about 1:5 to about 5:1, more preferably about 1:3, to about 1:1, depending upon the end use of the emulsion composition. The use of mineral oil in the present invention may be preferred. The inclusion of petrolatum and/or mineral oil and/or the ratio of petrolatum to mineral oil in the present compositions may influence the final viscosity of the water-in-oil compositions according to the present invention.

The term “secondary emulsifier” or “helper emulsifier” is used throughout the specification to describe compounds which are added to the emulsifier compositions according to the present invention to provide a more stable and in some embodiments consistent oil-in-water emulsion composition. Secondary or helper emulsifiers may be advantageous when formulating oil-in-water emulsion compositions which utilize one or more of the surfactants/emulsifiers according to the present invention. Secondary emulsifiers as used in an oil-in-water aspect of the present invention generally are considered surfactants which exhibit good surface activity and produce a low interfacial tension in the system in which it is used. Secondary emulsifiers preferably used in the present oil-in-water emulsions exhibit a tendency to migrate to the interface, rather than remain dissolved in either one of the water or oil phase. Mixtures of secondary emulsifiers actually may be preferred in certain embodiments, where the need is to provide better interaction between the oil and water phases. Secondary emulsifiers may be advantageously used in the present invention where the oil is a synthetic ester or a more polar oil. One of ordinary skill in the art may readily determine the type of secondary emulsifier or emulsifying system (group of emulsifiers) which may be used in the oil-in-water emulsions according to the present invention. A secondary emulsifier is used in the present invention in an amount effective to aid or promote emulsification of the water phase and oil phase (“emulsification effective amount”). As a general rule, the amount of secondary emulsifier which is included in compositions according to the present invention ranges from about 0.01% to about 7.5% or more by weight, more preferably about 0.1% to no more than about 5.0% by weight of the final emulsion composition. In oil-in-water emulsion compositions according to the present invention, where secondary emulsifiers are optionally included, the weight ratio of the present polyoxyethylene surfactants/emulsifiers according to the present invention to secondary emulsifier ranges from about 20:1 to about 1:20, more preferably about 10:1 to about 1:1.

Exemplary secondary emulsifiers for use in oil-in-water emulsions according to the present invention may be any cosmetically acceptable oil soluble non-ionic or anionic (and in certain instances quaternary or amphoteric) emulsifier/surfactant which has a hydrophilic group (“tail”) at one end of the molecule. Representative nonionic emulsifiers/surfactants for use as possible secondary emulsifiers in the present invention include, for example, glycereth-7 caprate/caprylate, PEG-6 caprylic/capric glycerides, PEG-7 glyceryl cocoate, glyceride esters, polyglyceride esters, ethoxylated fatty alcohols, ethoxylated fatty acids, ethoxylated fatty amines, ethoxylated fatty amides, ethoxylated fatty triglycerides, sucrose esters, alkyl glucosides, alkyl polyglucosides, fatty alcohols and fatty amides, among numerous others. Representative anionic emulsifers/surfactants for use as possible secondary emulsifiers include, for example, fatty acids, alkyl sulfates, alkylether sulfates, alkenyl sulfonates, alkyl phosphates, alkoyl arcosinates, alkyl sulfosuccinates, alkyl carboxylates, alkylether carboxylates, alkylamidoether carboxylates, trideceth-7 carboxylic acid, coceth-7 carboxylic acid and sodium dioctylsulfosuccinate, among numerous others. Representative cationic emulsifiers/surfactants which can be used here include, for example, fatty trimethylammonium chlorides, fatty trimethylammonium methosulfates, fatty dimethylethylammonium ethosulfate, fatty dimethylammonium salts, fatty amidopropyltrimethylammonium chlorides, fatty amidopropyltrimethylammonium methosulfates, fatty amidopropyldimethylethylammonium ethosulfate, fatty amidopropyldimethylammonium salts and soyamidopropyl ethyldimonium ethosulfate, among others. One of ordinary skill will understand to include one or more secondary emulsifiers in emulsion compositions according to the present invention in order to facilitate and enhance interaction of the water and oil phases.

The term “solvent” is used to describe an optional additive to the compositions according to the present invention which may be used to influence the rheology of the final composition and to help compatabilize components which may be added to compositions according to the present invention. The term solvent refers to any cosmetically acceptable or pharmaceutically compatible solvent, when used in context, and preferably refers to alcohol-type solvents. When used, solvents comprise from about 0.05-0.1% to about 20% by weight of the final composition, preferably about 1% to about 10%, more preferably about 2% to about 5%, more preferably about 2% to about 3% within this range, depending upon the oil, surfactant or emulsifier and amount of water used, the type of solvent to be used, as well as the solubility or physical characteristics of other components included in the final emulsions and final personal care compositions or other compositions. Preferred solvents for use in the present invention include, for example, ethoxydiglycol, propylene glycol, glycerine, butylene glycol, methylpropane diol (MP diol), hexylene glycol, sorbitol, ethanol, n-propanol, isopropanol, n-butanol, sec-butyl alcohol and tert-butyl alcohol, among others. It is noted that at higher concentrations of solvent, dilatancy characteristics of compositions of the present invention may diminish (even to the point of being non-existent depending upon the amount of solvent), or alternatively, the viscosity of the composition may simply diminish, regardless of shear.

The term “dilatant properties” or “dilatancy” is used throughout the specification to describe preferred rheological properties according to the present invention. Emulsions according to the present invention have dilatant properties or are characterized by dilatancy, i.e., these compositions become more viscous as increasing or greater shear forces are applied to the compositions. In other words, as shear forces are applied to preferred compositions according to the present invention, quite unexpectedly, the viscosity of the composition increases, rather than decreases, as is the convention. The shear forces may be as simple as the forces which occur as a composition is rubbed into the skin during application. Thus, in the present invention, compositions which are rubbed into the skin become perceptibly (by way of touch and/or feel) more viscous as force (rubbing) is applied to the compositions on the skin. In the present invention, when the compositions are rubbed into the skin, they become more viscous. This is counterintuitive, inasmuch as most compositions see a falloff or decrease in viscosity (sheer thinning) as shear forces are applied to the composition. In preferred aspects of the present invention, the viscosity (for example in centipoises (cps) units measured by any technique known in the art) increases as shear forces are applied to the composition, with certain preferred compositions actually doubling in viscosity as measured by a TA Instruments Rheometer, for example the AR series of rheometers—the AR-G2 or AR2000 (TA Instruments is in New Castle, Del., USA) using parallel serrated plates. The rheogram which is produced by a rheometer shows the response of the product in viscosity units between 0 and about 100,000 reciprocal seconds, preferably about 0.001 to 10,000 reciprocal seconds (shear rate). While low shear rates at 0.001-1 (0.01 to 1) reciprocal seconds will evidence a measurable (significant) difference in viscosity (between those measured at elevated shear rate versus those measured after standing or at 0 shear rate), greater shear rates will often cause an even greater increase in viscosity (although in certain instances, shear thinning, i.e., a decrease in viscosity even below standing viscosity, at high rates above about 10-100 reciprocal seconds may occur, depending upon composition). Preferred compositions according to the present invention have a viscosity, as measured in centipoises units, under shear force or shear conditions within the range of about 0.001-1 (preferably about 0.01-1) reciprocal seconds as defined above, which is at least about 1.5 times the viscosity, at least about 2.0 times the viscosity, at least about 2.5 times the viscosity, at least about 3.0 times the viscosity, at least about 5 times the viscosity, at least about 10 times the viscosity, at least about 25 times the viscosity, at least about 50 times the viscosity, at least about 100 times the viscosity, at least about 500 times the viscosity, at least about 1000 times the viscosity or more than the viscosity of the composition which is not under shear force (e.g., after the composition has settled or is stored for a sufficient period—at rest). In certain instances, the greatest viscosity for many of the compositions will occur at a shear rate of about 1 reciprocal second. Viscosity measurements also may be obtained by using a standard Brookfield viscometer or any other viscometer which may be standard in the art. For example, viscosity measurements may be made using a Brookfield LVT viscometer (preferably using spindle #3) and shear rates determined accordingly.

The current invention relates to the use of unique, dilatant emulsions which are produced using specific nonionic polymeric emulsifiers. These compounds/compositions are represented by the following three chemical formulas I, II and III: 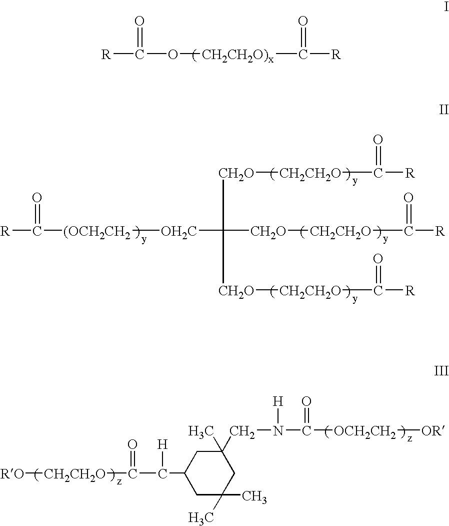
wherein x is about 150; y is about 35 to about 40 such that the total of 4y is about 150 and z is about 75-100, preferably 75 or 100; and R is a C ₁₅-C₂₁ alkyl or alkene group, preferably an alkyl group, more preferably a C₁₇ alkyl group; and R′ is a C₁₆-C₂₂ alkyl or alkene group, preferably an alkyl group, more preferably a C₁₈ alkyl group.

The emulsifiers as described herein may be used to produce emulsions, said emulsion comprising an oil in an amount ranging from about 5% to about 90% by weight (preferably about 60% to about 85% of an oil), water in an amount ranging from about 10% to about 95% by weight water and an emulsifier selected from one or more of the compounds/compositions according to any one or more of formulas I, II or III in an amount ranging from about 0.1% to about 20% by weight of an emulsifier (preferably about 1% to about 10% of an emulsifier, even more preferably about 1% to about 5% of an emsulsifier within this range which varies as a function of the amount of oil and water within the composition), and optionally, about 0% to about 10% (preferably, at least about 0.05-0.1% to about 5% within this range) by weight of a secondary emulsifier and about 0% to about 20% by weight of a solvent (preferably, about 0.1% to about 10% by weight, preferably, about 2% to about 5% by weight), depending upon solvent and the amount of oil, water, emulsifier and type of emulsifier used in the composition noting that higher weight % of solvent may negatively impact the dilatancy characteristics of the composition, making the composition thinner and less viscous as the weight percent of solvent increases. In certain embodiments, emulsions according to the present invention may be adapted as personal care products or topical delivery systems for bioactives. In the case of personal care products, emulsions according to the present invention may be adapted to include effective amounts of coloring agents, secondary emulsifiers, pigments, anti-perspirant agents, fragrances, aromatic or deodorizing agents, uv-absorbing compounds (for use in sunscreens), preservatives, skin and/or hair conditioning agents, hair-setting polymers, and hair-straightening agents, among numerous others. Particular additives include, for example, solid materials such as, but not limited to, talc, silica, alumina, pigments, solid organic polymers and waxes, which may be included in compositions according to the present invention in effective amounts. These solid materials may influence the dilatent properties of emulsions and final personal care compositions according to the present invention.

These compounds may be added to the basic components of the emulsions of the present invention in amounts ranging from about 0.01% to about 25% by weight or more, depending upon the nature of the final emulsion composition and final composition in which the emulsion is used.

The compounds of formula III are the preferred emulsifiers of the present invention and are urethane dimers that are formed by the reaction of two moles of ethoxylated fatty alcohol with one mole of isophorone diisocyanate. The preferred examples of this invention embody fatty alcohols that are highly ethoxylated, having from about 75 to 100 oxyethylene units reacted onto the fatty alcohol. Commercially, these materials find use as viscosity builders and are sold for that purpose, similar to PEG-150 distearate. Now, it has been surprisingly found that these materials also function as emulsifiers. While it has been known that materials such as PEG-150 Distearate function as emulsifiers in addition to building viscosity, these compounds are not thought to be efficient emulsifiers and generally find use only as secondary emulsifiers and emulsion stabilizers. We have unexpectedly found that these surfactants may be used to produce emulsifiers exhibiting dilatancy.

Emulsions according to the present invention may be made preferably by forming a mixture of water and emulsifier (which may be at elevated temperature) and then adding the oil to the water/surfactant mixture at elevated temperature, preferably at least about 75° C., more preferably between about 80 and 100° C. The emulsion composition should be quickly (i.e., without delay), but gently mixed, preferably at propeller mixing speeds of less than 100 rpm, noting that as the admixture becomes homogeneous and forms an emulsion, as mixing speeds increase, the dilatancy of the emulsion compositions will be magnified, thus complicating production. Additional components are preferably added to the water phase or the oil phase before mixing (depending upon the physicochemical characteristics of the component) adding, in any order, the oil, water and emulsifier and optional ingredients, if used, to produce an initial mixture and then mixing the mixture to homogeneity. It is noted that upon the emulsion becoming homogeneous, it will become viscous under shear, which may complicate further mixing, especially if strong shear forces are being used for mixing. Secondary emulsifiers and/or solvent may be added either before mixing or after mixing occurs. In the case of the addition of a secondary emulsifier, this may enhance the production of an emulsion. Solvent, in an amount ranging up to about 20%, may be used to reduce the viscosity of the composition, to facilitate mixing and to reduce the dilatancy of the final composition.

The emulsions of the present invention may be combined with other components to produce personal care products. These components may be added at the time of the initial mixing, preferably by adding individual componens to either the water/surfactant phase or the oil phase (depending upon the solubility characteristics of the components to be added), and then mixing at elevated temperatures (as described above) gently to promote homogeneity. Alternatively, the components may be added after the formulation of the emulsion at elevated temperature, preferably at least about 75° C., more preferably between about 80 and 100° C.

Having generally described the invention, reference is now made to the following examples which are intended to illustrate preferred embodiments and comparisons but which are not to be construed as limiting to the scope of this invention as is more broadly set forth above and in the appended claims. The following examples illustrate the intended uses for the present emulsifiers, describe the properties of the emulsions and present some practical product applications.

| | 1 | 2 | 3 | 4 |
|---|---|---|---|---|
| Ingredients | % | % | % | % |
| Mineral Oil (70 ssu) | 50.0 | 50.0 | 50.0 | 50.0 |
| PEG-150 Distearate | 3.5 | — | — | — |
| PEG-150 Pentaerythrityl Tetrastearate | — | 3.5 | — | — |
| Disteareth-75 IPDI | — | — | 3.5 | — |
| Disteareth-100 IPDI | — | — | — | 3.5 |
| Deionized Water | 45.5 | 45.5 | 45.5 | 45.5 |
| Phenobact | 1.0 | 1.0 | 1.0 | 1.0 |
| | 100.0 | 100.0 | 100.0 | 100.0 |

Example 1 shows the use of PEG-150 Distearate as an emulsifier. Surprisingly, it is very efficient, having an emulsifier to oil phase ratio of 7% as compared to typical nonionic emulsions that have emulsifier to oil phase ratios of 10% to 25%. The emulsion produced is a viscous, white lotion that is shear thinning. See Chart 1 for details of viscosity and rheology.

Example 2 shows the use of PEG-150 Pentaerythrityl Tetrastearate as an emulsifier. It is also very efficient and has an emulsifier to oil phase ratio of 7%. The emulsion produced is a viscous, white lotion that is free flowing and dilatant.

Example 3 shows the use of Disteareth-75 IPDI as an emulsifier. Again, it is very efficient, having an emulsifier to oil phase ratio of 7%. The emulsion produced is a viscous, white lotion that is only very slowly free flowing under low shear (i.e. gravity) and is very dilatant becoming almost rubbery when prodded with a finger and resisting flow to such a degree that it fractures into crumbly pieces, when vigorously shaken in a jar.

Example 4 shows the use of Disteareth-100 IPDI as an emulsifier. It is an efficient emulsifier with an oil to emulsifier ratio of 7%. This emulsion is quite similar in appearance to the PEG-150 Pentaerythrityl Tetrastearate emulsion of example 2. The emulsion produced is a viscous, white lotion that is free flowing and dilatant.

The unique and defining property of dilatant emulsions is that they are increasingly resistant to flow at increasing shear rates. That is, they are “shear thickening”. This is as opposed to typical cosmetic emulsions that are “shear thinning” and flow more readily with increasing shear rate. Thus, a dilatant emulsion may flow easily if its container is simply tilted and the product is allowed to flow under the influence of gravity, but it will gel and not flow at all if an attempt is made to force the emulsion out by shaking it. Some dilatant emulsions resist high shear so strongly that they fracture into pieces when rubbed between the hands or upon a surface or when shaken vigorously in a container. Once the high shear force is stopped and low shear, such as gravity reasserts, the fractured emulsion pieces will flow, as if melting. As one would expect, there are varying degrees of “dilatantcy”. Thus, formulations can be, made that will flow even when subjected to high shear, although their viscosity is higher than the “at rest” viscosity. Conversely, formulations are available that are stiff, non-flowing gels even when at rest. Example 3 shows flow characteristics that are typical of a very dilatant emulsion—in that it is a stiff emulsion that does not flow when at rest and fractures into crumb-like pieces when vigorously shaken in a jar.

Emulsions such as Examples 2 and 4 exhibit viscous flow and can be poured from a container, but they become very “rubbery” when prodded with a finger and they do not readily adhere to the finger-making them difficult to “pick up” from a jar. Spreading these emulsions on the skin can also be problematic, as they will “ball up” when rubbed. So, at times, some persistence is required to apply them, but they do eventually spread and, as the emulsion looses water, they spread much more easily. Interestingly, the dilatant emulsions made with the emulsifiers of the present invention, spread relatively easily on skin, because (for whatever reason) they appear to readily deposit the internal oil phase when rubbed upon the skin.

The addition of alcohols, glycols, hydrotropes and other solvents can be used to modify the viscosity and rheology of these systems. Thus, the physical properties can be varied to suit specific needs. Also, other surfactants can be added to modify the ease with which an emulsion is formed and the rheology of the resulting emulsions or the intended use of the emulsion. Further, the emulsifier materials of this invention can be used to make emulsions with more typical oil phase concentrations, in which case, the emulsions have more typical properties but, they remain dilatant. The type of oil that is emulsified can also be varied and includes, but is not limited to mineral oils, triglycerides, silicone based fluids, UV absorbers and polymers. Finally, materials such as viscosity controlling polymers, film forming polymers, slip and feel modifying ingredients, fragrances, preservatives, vitamins, medicaments and colorants can be added to modify the emulsions and vary their intended use. Some examples of these additions are described in the following examples.

| | 5 | 6 | 7 | 8 |
|---|---|---|---|---|
| Ingredients | % | % | % | % |
| Mineral Oil (70 ssu) | 50.00 | — | 5.00 | — |
| Disteareth-75 IPDI | 3.50 | 3.50 | 3.25 | 2.00 |
| Disteareth-100 IPDI | — | — | — | 1.00 |
| Coceth-7 Carboxylic acid | 1.75 | — | 1.73 | — |
| Propylene Glycol | 10.00 | — | — | — |
| Soyamidopropyl Ethyldimonium | — | 1.75 | — | — |
| Ethosulfate | | | | |
| Meadowfoamseed Oil | — | 3.00 | — | — |
| Coconut Oil | — | 3.00 | — | — |
| Wheatgerm Oil | — | 3.00 | — | — |
| Brazilnut Oil | — | 3.00 | — | — |
| Dipentaerythritol | — | — | 6.00 | — |
| Tetrahydroxystearate/Isostearate | | | | |
| Diglyceryl Diisostearate | — | — | 2.00 | — |
| Polyurethane 18 | — | — | 5.00 | — |
| Dimethicone (100 cps) | — | — | — | 80.00 |
| Deionized Water | 33.75 | 81.75 | 76.02 | 16.00 |
| Phenobact | 1.00 | 1.00 | 1.00 | 1.00 |
| | 100.00 | 100.00 | 100.00 | 100.00 |

Example 5 shows the use of Disteareth-75 IPDI with an auxiliary anionic surfactant. The surfactant helps to solubilize the Disteareth-75 IPDI which gels when it is placed in water by itself and becomes insoluble. In a separate experiment, it was found that the Coceth-7 Carboxylic acid alone did not make an emulsion. The emulsion formed immediately upon addition of the hot (80-85° C.) mineral oil to the hot (80-85° C.) water plus emulsifier mixture. The initial emulsion was a white cream/gel. The addition of the propylene glycol reduced the dilatantcy although not eliminating it. This formulation would be suitable for a hand and body cream or for a hair pomade. Example 6 shows a hair conditioner formula that has an oil level of 12% total, which is a more typical formulation amount. In this instance, the Disteareth-75 IPDI is solulibized with Soyamidopropyl Ethyldimonium Ethosulfate. This surfactant is cationic and, in addition to helping to dissolve the Disteareth-75 IPDI, it also is a hair conditioner that improves the wet combability, and the static electricity control of the hair. In separate experiments, it was determined that the emulsion forms in the absence of the Soyamidopropyl Ethyldimonium Ethosulfate, but not in the absence of the Disteareth-75 IPDI.

Example 7 is a hair setting pomade/gel that includes Polyurethane 18 as the set holding resin. The mineral oil and the isostearate esters provide shine and manageability. This is a dilatant emulsion and provides a unique usage experience. Example 8 shows the use of the more water soluble Disteareth-100 IPDI as the emulsifier /solubilizer for the Disteareth-75 IPDI. The formula also demonstrates the ability of the Disteareth-75 IPDI and Disteareth-100 IPDI materials to emulsify materials such as dimethicone. This emulsion is dilatant and suitable for use as a hair styling aid or a hand cream. Note that the dimethicone concentration is very high. This is not unusual for these types of emulsifiers and yet they are oil-in-water emulsions as shown by their ability to conduct electricity, their dispersability in water, and their ready absorption of colored water (dilute solution of an anionic food color dye in water). Further examples of high internal phase emulsions are given in the following Examples.

| | 9 | 10 | 11 | 12 |
|---|---|---|---|---|
| Ingredients | % | % | % | % |
| Mineral Oil (70 ssu) | 70.0 | 80.0 | 80.0 | 90.0 |
| PEG-150 Distearate | 3.5 | — | — | — |
| PEG-150 Pentaerythrityl Tetrastearate | — | 3.5 | — | — |
| Disteareth-100 IPDI | — | — | 3.5 | 1.0 |
| Deionized Water | 26.5 | 16.5 | 16.5 | 9.0 |
| | 100.0 | 100.0 | 100.0 | 100.0 |

Example 9 shows the use of PEG-150 Distearate as an emulsifier with an emulsifier to oil phase ratio of 5%. The emulsion produced is a solid, non-flowing white emulsion that also resists flowing under high shear stress and has a characteristic “ring”.

Example 10 shows the use of PEG-150 Pentaerythrityl Tetrastearate as an emulsifier in a high internal phase (80%) oil-in-water emulsion. This is a dilatant, “rubbery” emulsion that also “rings” when tapped.

Example 11 shows the use of Disteareth-100 IPDI as an emulsifier in a high internal phase (80%) oil-in-water emulsion. This is a very dilatant, crumbly emulsion that “rings”when tapped and does not flow under the influence of gravity.

The absence of PEG-150 Distearate and Disteareth-75 IPDI from the chart above is also of interest. Neither of these two materials made acceptable emulsions at 3.5% and 80% mineral oil. The PEG-150 Distearate emulsion did not form and the Disteareth-75 IPDI did not emulsify all of the oil. The limits on the emulsifying ability of these two materials appear to be due to the concentration of the emulsifier as can be seen in the next Example.

Example 12 shows the use of Disteareth-100 IPDI as an emulsifier in a high internal phase (90%) oil-in-water emulsion. This is a dilatant emulsion that also “rings” when tapped. The emulsion is quite stiff and crumbly. It also shows electrical conductivity indicating that the emulsion is oil-in-water.

| | 13 | 14 | 15 |
|---|---|---|---|
| Ingredients | % | % | % |
| Mineral Oil (70 ssu) | 90.0 | 90.00 | — |
| Petrolatum | — | — | 50.00 |
| PEG-150 Distearate | 1.0 | — | — |
| Disteareth-75 IPDI | — | 1.00 | — |
| Disteareth-100 IPDI | — | — | 4.25 |
| Mackanate DOS-70 | — | 0.15 | — |
| Propylene Glycol | — | — | 3.48 |
| Sodium Hydroxide (50% aq. soln.) | — | — | 4.00 |
| Deionized Water | 9.0 | 8.85 | 45.50 |
| | 100.0 | 100.00 | 100.00 |

Example 13 shows the use of PEG-150 Distearate as an emulsifier for an emulsion containing a very high oil level. It also conducts electricity showing it to be an oil-in-water emulsion. The emulsion is dilatant, but it is softer than Example 12, it is also not crumbly and does not ring when the container is tapped. Example 14 shows a very high oil content emulsion using Disteareth-75 IPDI and an auxiliary surfactant to help solubilize/disperse the Disteareth-75 IPDI. This emulsion is very dilatant and also rings. Disteareth-75 IPDI and PEG-150 Pentaerythrityl Tetrastearate are not as water soluble as Disteareth-100 IPDI or PEG-150 Distearate and, in general, require assistance, in the form of auxiliary surfactants, in order to produce acceptable emulsions. But, again in general, the amounts of Disteareth-75 IPDI or PEG-150 Pentaerythrityl Tetrastearate used to achieve emulsification are less than either Disteareth-100 IPDI or PEG-150 Distearate and the dilatantcy and ringing effects observed in their emulsions are usually more pronounced. Example 15 shows a practical application of this technology in the form of a hair relaxer. The emulsion is a smooth, viscous flowing, dilatant, but non-ringing, white cream or heavy lotion that is suitable for packaging in a tube or jar. The advantage of this formulation is the extreme hydrolytic stability of the Disteareth-100 IPDI. Thus, there is very little degradation of the formula over time or with elevated temperature storage.

CHART 1
Brookfield LVT Viscometer

| | | |
|---|---|---|
| Example # 1 | Viscosity - Spindle # 3 | Viscosity Spindle # 4 |
| | 31,600 @ 3.0 rpm | 30,000 @ 6.0 rpm |
| | 38,400 @ 1.5 rpm | 36,000 @ 3.0 rpm |
| | 54,000 @ 0.6 rpm | |
| | 72,000 @ 0.3 rpm | |
| Example # 2 | Viscosity - Spindle # 3 | Viscosity Spindle # 4 |
| | 60,800 @ 1.5 rpm | 48,000 @ 6.0 rpm |
| | 162,000 @ 0.6 rpm | 58,000 @ 3.0 rpm |
| | 104,000 @ 0.3 rpm | 70,600 @ 1.5 rpm |

| | |
|---|---|
| Example # 3 | discontinuous gel - unable to obtain |
| | stable readings |

| | | |
|---|---|---|
| Example # 4 | Viscosity - Spindle # 3 | Viscosity Spindle # 4 |
| | 67,200 @ 1.5 rpm | 56,500 @ 6.0 rpm |
| | 80,000 @ 0.6 rpm | 67,000 @ 3.0 rpm |
| | 90,000 @ 0.3 rpm | 78,000 @ 1.5 rpm |

Amendments to the claims:

1. An emulsion composition comprising:
i. about 5% to about 90% by weight of an oil;
ii. about 10% to about 95% by weight water;
iii. about 0.1 % to about 20% by weight of at least one emulsifier according to chemical formulas I, II or III: 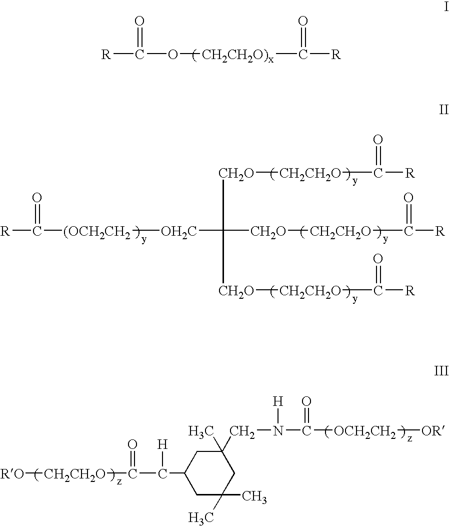
wherein x is about 150; y is about 35 to about 40 such that the total of 4y is about 150 and z is about 75-100; and
Each R is independently a C₁₅-C₂₁alkyl or alkene group; and
Each R′ is independently a C₁₆-C₂₂ alkyl or alkene group;
iv. optionally, about 0.1% to about 20% by weight of a cosmetically or pharmaceutically compatible solvent; and
v. about 0% to about 10% by weight of a secondary emulsifier.

2. The composition according to claim 1 wherein said emulsifier comprises about 1% to about 10% by weight.

3. There composition according to claim 1 wherein said emulsifier is a compound according to formula mi and wherein z is 75 or 100.

4. The composition according to claim 1 wherein R is an alkyl group.

5. The composition according to claim 1 wherein R′ is an alkyl group.

6. The composition according to claim 1 wherein R and R′ are both alkyl groups.

7. The composition according to claim 1 wherein R is a C₁₇ alkyl group.

8. The composition according to claim 1 wherein R′ is a C₁₈ alkyl group.

9. The composition according to claim 1 wherein R is a C₁₇ alkyl group and R′ is a C₁₈ alkyl group.

10. The composition according to claim 1 wherein said emulsifier is a compound according to formula II or III and R and R′ are both alkyl groups.

11. The composition according to claim 1 wherein said solvent is selected from the group consisting of ethoxydiglycol, propylene glycol, glycerine, butylene glycol, methylpropane diol, hexylene glycol, sorbitol, ethanol, n-propanol, isopropanol, n-butanol, sec-butyl alcohol, tert-butyl alcohol and mixtures thereof.

12. The composition according to claim 1 wherein said solvent is ethanol, glycerine, propylene glycol, butylene glycol, MPdiol or sorbitol.

13. The composition according to claim 1 wherein said oil is selected from the group consisting of polar and non-polar oils.

14. The composition according to claim 13 wherein said oil is selected from the group consisting of mineral oil, petrolatum and mixtures, thereof.

15. The composition according to claim 1 wherein said oil is a glyceride ester.

16. The composition according to claim 1 wherein z is 75.

17. The composition according to claim 1 wherein z is 100.

18. The composition according to claim 3 wherein z is 75.

19. The composition according to claim 3 wherein z is 100.

20. The composition according to claim I wherein said solvent is a hydrotrope.

21. The composition according to claim 1 further comprising at least one component selected from the group consisting of talc, silica, alumina, pigments, solid organic polymers, waxes and mixtures thereof.

22. The composition according to claim 1 wherein said oil comprises at least about 70% by weight.

23. A composition according to claim 1 wherein said composition exhibits dilatancy properties.

24. A personal care composition comprising the emulsion of claim 1 in combination with at least one additional component selected from the group consisting of talc, silica, alumina, pigments, solid organic polymers, waxes, coloring agents, humectants, moisturizing agents, anti-perspirant agents, aromatic agents, deodorizing agents, uv-absorbing compounds, preservatives, skin and/or hair conditioning agents, hair-straightening agents and mixtures thereof.

25. A method of producing an emulsion comprising:
i. adding an emulsifier to water and mixing to form a water/emulsifier mixture;
ii. adding an oil to said water/emulsifier mixture to form an admixture; and
iii. mixing said admixture at elevated temperature to produce an emulsion composition;
wherein said emulsion composition comprises:
a. about 5% to about 90% by weight of an oil;
b. about 10% to about 95% by weight water; and
c. about 0.1 % to about 20% by weight of at least one emulsifier according to chemical formulas I, II or III: 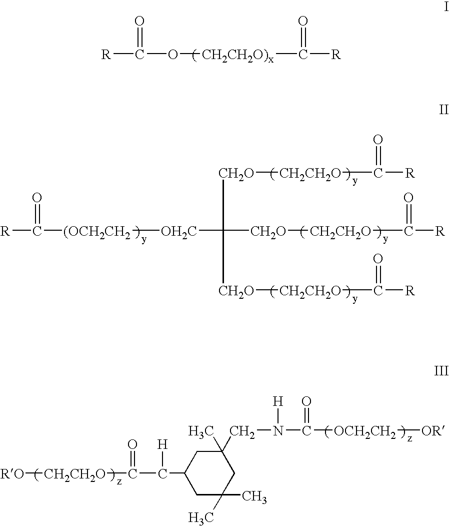
wherein x is about 150; y is about 35 to about 40 such that the total of 4y is about 150 and z is about 75-100;
Each R is independently a C₁₅-C₂₁alkyl or alkene group; and
Each R′ is independently a C₁₆-C₂₂ alkyl or alkene group;

26. (canceled)

27. (canceled)

28. (canceled)

29. (canceled)

30. (canceled)

31. (canceled)

32. (canceled)

33. (canceled)

34. The menthod according to claim 25 wherein said emulsion exhibits dilatancy properties.
